(19) United States
(12) Patent Application Publication
(10) Pub. No.: US 2010/0303887 A1
(43) Pub. Date: Dec. 2, 2010

(54) DHA and PEDF, a Therapeutic Composition for Nerve and Retinal Pigment Epithelial Cells

(76) Inventor: Nicolas G. Bazan, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/670,986

(22) PCT Filed: Jul. 30, 2008
(86) PCT No.: PCT/US2008/071577

§ 371 Date: Jul. 23, 2010

Related U.S. Application Data
(60) Provisional application No. 60/952,725, filed on Jul. 30, 2007.

(51) Int. Cl. A61K 9/00; A61K 38/22; A61P 27/02; A61P 25/00

(52) U.S. Cl. 424/424; 514/9.7; 424/422; 424/423

(57) ABSTRACT

The combination of pigment epithelium-derived factor (PEDF) and docosahexaenoic acid (DHA) has been discovered to act synergistically to enhance cell survival and decrease apoptosis in retinal pigment epithelial (RPE) cells. PEDF and DHA synergistically protected RPE cells by confronted with oxidative stress by blocking apoptotic cell death and increasing the synthesis of the important mediator neuroprotectin D 1. Administering a composition comprising PEDF and DHA will halt or slow down the initiation and progression of macular degeneration, retinitis pigmentosa and retinal degeneration. In addition, the topical application of the combination of PEDF and DHA was found to promote cornea nerve regeneration after refractive surgery, and thus this combination could be used to prevent the complications of refractive surgery and certain diseases, e.g., neurotrophic keratitis due to Herpes virus.

(In countries other than the United States:) The benefit of the 30 Jul. 2007 filing date of U.S. patent application Ser. No. 60/952,725 is claimed under applicable treaties and conventions. (In the United States:) The benefit of the 30 Jul. 2007 filing date of provisional patent application No. 60/952,725 is claimed under 35 U.S.C. §119(e).

The development of this invention was partially funded by the Government under grant number EY05121 from the National Institutes of Health National Eye Institute, and grant number P20 RR016816 from the National Institutes of Health National Center for Research Resources. The Government has certain rights in this invention.

### TECHNICAL FIELD

This invention pertains to the use of the combination of pigment epithelium-derived factor (PEDF) and docosahexaenoic acid (DHA) to protect the survival of nerve cells and retinal pigment epithelial (RPE) cells. This unique combination acts synergistically in nerve cells and RPE cells to increase the local concentration of neuroprotectin 1 (NPD1) and inhibit apoptosis. In addition, topical administration of this unique combination using a collagen shield was shown to promote the regeneration of corneal nerves after experimental refractive surgery.

### BACKGROUND ART

### Docosanoids

Dietary omega-3 fatty acids are required to maintain cellular functional integrity, and overall are necessary to human health. Docosahexaenoic acid (22:6, n-3, DHA), a major component of fish oil and marine algae, is highly concentrated in photoreceptors, brain and retinal synapses. See, Bazan, N. G. (1990) in Nutrition and the Brain, vol. 8, eds. Wurtman, R. J. & Wurtman, J. J., (Raven Press, Ltd., New York) pp. 1-24. Diet-supplied DHA or its precursor (18:3, n-3) are initially taken up by the liver and then distributed through blood lipoproteins to meet the needs of organs, notably during photoreceptor cell biogenesis and synaptogenesis. See, Scott, B. L. & Bazan, N. G. (1989) Proc. Nail. Acad. Sci. U.S.A. 86:2903-2907. DHA has been reported to be involved in memory-related learning ability, excitable membrane function, photoreceptor cell biogenesis and function, and signal transduction pathways in which protein kinases are involved. See, Gamoh, S., Hashimoto, M., Sugioka, K., Shandat Hossain, M., Hata, N., Misawa, Y., and Masumura, S. (1999) Neuroscience 93, 237-241; McGahon, B. M., Martin, D. S., Horrobin, D. F., and Lynch, M. A. (1999) Neuroscience 94, 305-314; Gordon, W. C., and Bazan, N. G. (1990) J. Neurosci. 10, 2190-2202; Miniikjoo, B., Brown, S. E., Seung Kim, H. F., Marangell, L. B., Sweatt, J. D., and Weeber, E. J. (2001) J. Biol. Chem. 276, 10888-10896. DHA has also been implicated in protecting nerve cells from apoptotic cell death as a membrane component, and other neuroprotective bioactivity. See, Kim, H.-Y., Akbar, M., Lau, A., and Edsall, L. (2000) J. Biol. Chem. 275, 35215-35223; Lauritzen, I., Blondeau, N., Heurteaux, C., Widmann, C., Romey, G., and Lazdunski, M. (2000) EMBO J. 19, 1784-1793; and Rodriguez de Turco, E. B., Belayev, L., Liu, Y., Busto, R., Parkins, N., Bazan, N. G., and Ginsberg, M. D. (2002) J. Neurochem. 83, 515-524. Whether DHA itself or a DHA-derived messenger is involved in these events is not known. DHA was also found decreased in the hippocampus of aged rats. (McGahon et al., 1999) Moreover, to date potent bioactive autacoids from DHA acting in nanomolar concentrations have not been identified in the central nervous system. Although certain docosanoids have been identified in retina, their physiologic properties have not been explored. See, Bazan, N. G., Birkle, D. L., and Reddy, T. S. (1984) Biochem. Biophys. Res. Commun. 125, 741-747; and Serhan, C. N., Clish, C. B., Brannon, J., Colgan, S. P., Chiang, N. & Gronert, K. (2000) J. Exp. Med. 192:1197-1204.

Retinal Pigment Epithelial Cells and Retinal Diseases

Photoreceptor outer segments contain rhodopsin as well as the highest content of DHA of any cell type. In contact with the photoreceptor tips is a monolayer of cells, the retinal pigment epithelium (RPE), derived from neuroepithelium. These cells are the most active phagocytes of the body. In a daily cycle, they engulf and phagocytize the distal tips of photoreceptor outer segments, thereby participating in rod outer segment renewal in a process that is balanced by addition of new membrane to the base of the outer segments. The conservation of DHA in photoreceptors is supported by retrieval through the interphotoreceptor matrix, which supplies the fatty acid for the biogenesis of outer segments. See, Stinson, A. M., Wiegand, R. D. & Anderson, R. E. (1991) J. Lipid Res. 32:2009-2017; Bazan, N. G., Birkle, D. L. & Reddy, T. S. (1985) in Retinal Degeneration: Experimental and Clinical Studies. Eds. LaVail, M. M., Anderson, R. E., & Hollyfield, J. (Alan R. Liss, Inc., New York) pp. 159-187; and Gordon, W. C., Rodriguez de Turco, E. B. & Bazan, N. G. (1992) Curr. Eve Res. 11:73-83. The continuous renewal of photoreceptors is tightly regulated so that their length and chemical composition, including that of their phospholipids, are maintained. Photoreceptor phospholipids contain most of their DHA in carbon 2 of the glycerol backbone. However, they also display molecular species of phospholipids containing DHA in both C1 and C2 positions of the glycerol backbone, as well as polyunsaturated fatty acids of longer chains than C22 that result from subsequent elongation of DHA. See, Choe, H-G & Anderson, R. E. (1990) Exp. Eye Res. 51:159-165. Retina, as well as brain, displays an unusual DHA-retention ability, even during very prolonged dietary deprivation of essential fatty acids of the omega-3 family. In fact, to effectively reduce the content of DHA in retina and brain in rodents and even in non-human primates, dietary deprivation for more than one generation has been necessary. Under these conditions, impairments of retinal function have been reported. See, Wheeler, T. G., Benolken, R. M. & Anderson, R. E. (1975) Science. 188:1312-1314; and Neuringer, M., Connor, W. E., Van Petten, C. & Barstad, L. (1984) J. Clin. Invest. 73:272-276.

DHA is highly concentrated as an acyl group of phospholipids in photoreceptor outer segment disc membranes. The RPE cell actively recycles DHA from phagocytized disc membranes back to the inner segment of the photoreceptor cell. In addition, the RPE cell takes up DHA from the blood stream through the choriocapillaris. The RPE cell thus is very active in the uptake, conservation, and delivery of DHA. The apical side of the RPE cell participates in the recognition and shedding of photoreceptors during outer segment phagocytosis. See, Rattner, A. (2006), Nat. Rev. Neurosci. 7:860-872; and Bazan, N. G. (2006) Trends Neurosci. 29:263-271.

The high content of DHA in photoreceptor and RPE cells has to date been linked mainly to endowing photoreceptor membrane domains with physical properties that contribute to the modulation of receptors (e.g., rhodopsin), ion channels, transporters, etc. For example, in other cells DHA modulates G-protein-coupled receptors and ion channels. Moreover, DHA has been suggested to regulate membrane function by maintaining its concentration in phosphatidylserine. See, Salem, N. Jr., Litman, B., Kim, H. Y. & Gawrisch, K. (2001) Lipids. 36:945-959; and Gu, X., Meer, S. G., Miyagi, M., Rayborn, M. E., Hollyfield, J. G., Crabb, J. W., and Salomon, R. G. (2003) J. Biol. Chem. 278: 42027-42035. DHA is also envisioned as a target of oxidative stress, mainly by reactive oxygen intermediates that in turn trigger RPE and photoreceptor cell damage.

Rhodopsin mutations in retinitis pigmentosa expressed in rats are associated with a decreased content of DHA in photoreceptors. See, Anderson, R. E, Maude, M. B., McClellan, M., Matthes, M. T., Yasumura, D. & LaVail, M. M. (2002) Mol. Vis. 8:351-358. This observation is interpreted as a retinal response to a metabolic stress, whereby decreasing the amount of the major target of lipid peroxidation, DHA, elicits protection. Retinal degeneration induced by constant light promotes DHA loss from photoreceptors, but rats reared in bright cyclic light are protected. See, Li, F., Cao, W. & Anderson, R. E. (2001)Exp. Eve Res. 73:569-577. These studies suggest a remarkable adaptation/plasticity that may involve endogenous molecules that have not been characterized.

RPE cells also perform several other functions, including transport and reisomerization of bleached visual pigments, and contribute to the maintenance of the integrity of the blood-outer retinal barrier. Retinal detachment or trauma triggers dysfunctions in the RPE cells that lead to the onset and development of proliferative vitreoretinopathy.

RPE cells are essential for photoreceptor cell survival. When RPE cells are damaged or die, photoreceptor function is impaired, and the cells die as a consequence. Thus, oxidative stress-mediated injury and cell death in RPE cells impair vision, particularly when the RPE cells of the macula are affected. The macula is responsible for visual acuity. The pathophysiology of many retinal degenerations (e.g., age-related macular degenerations and Stargardt's disease) involves oxidative stress leading to apoptosis of RPE cells. In fact, RPE cell damage and apoptosis seem to be dominant factors in age-related macular degeneration. See, Hinton, D. R., He, S. & Lopez, P. F. (1998) Arch. Opthalmol. 116:203-209. In Stargardt's disease, the lipofuscin fluorophore A2E mediates RPE damage. The caspase-3 enzyme has been shown to be pail of this cascade, whereas the anti-apoptotic Bcl-2 protein exerts cellular protection. See, Sparrow, J. R. Vollmer-Snarr, H. R., Zhou, J., Jang, Y. P., Jockusch, S., Itagaki, Y. & Nakanishi, K. (2003) J. Biol. Chem. 278:18207-18213. RPE apoptosis precedes the demise of photoreceptors.

Oxidative stress triggers multiple signaling pathways. Some are cytoprotective, and others lead to cell damage and eventually cell death. Among these signals are the Bcl-2 family proteins. In fact, expression of pro- and anti-apoptotic Bcl-2 family proteins is altered by oxidative stress and represents a major factor, insofar as the outcome of the apoptotic signaling, since cell survival reflects the predominance of one set of proteins over the other. In the RPE and photoreceptor cells, oxidative stress, induced by several factors including retinal light exposure or reactive oxygen species, triggered an unfavorable shift in the Bcl-2 family proteins toward cell damage. See, Osborne, N. N., Cazevieillem C., Pergandem G. & Wood, J. P. (1997) Invest. Opthalmol. IIs. Sci. 38:1390-1400; and Liang, Y. G., Jorgensen, A. G., Kaestel, C. G., Wiencke, A. K., Lui, G. M., la Cour, M. H., Ropke, C. H. & Nissen, M. H.(2000) Curr. Eye Res. 20:25-34.

Retinal DHA is a target of oxidative stress-mediated lipid peroxidation. See, Organisciak, D. T., Darrow, R. M., Jiang, Y. L. & Blanks, J. C. (1996) Invest. Opthalmol. Vis. Sci. 37:2243-2257. Oxidative stress in brain generates neuroprostanes from DHA through an enzyme-independent reaction in brain, and in the retina DHA is thought to be an active site of lipid peroxidation. See, Roberts, L. J. 2nd, Montine, T. J., Markesbery, W. R., Tapper, A. R., Hardy, P., Chemtob, S., Dettbarn, W. D. & Morrow, J. D. (1998) J. Biol. Chem. 273:13605-13612. There are also studies that demonstrate DHA-mediated neuroprotection in photoreceptor cells and brain. See, Rotstein, N. P., Aveldano, M. I, Barrantes, F. J., Roccamo, A. M. & Politi, L. E. (1997) J. Neurochem. 69:504-513; and Rotstein, N. P. Politim, L. E., Germanm, O. L. & Girotti, R. (2003) Invest. Opthalmol. Vis. Sci. 44:2252-2259; and Kim, H. Y., Akbar, M., Lau, A. & Edsall, L. (2000) J. Biol. Chem. 275:35215-352. To date, no specific DHA mediators have been identified that elicit protection in RPE cells, except certain docosanoids identified in retina, the formation of which is inhibited by lipoxygenase inhibitors. Although the bioactivity of these docosanoids has not been studied, they were suggested to be neuroprotective. See, Bazan, N. G., Birkle, D. L. & Reddy, T. S. (1984) Biochem. Biophs. Res. Commun. 125:741-747; Bazan, N. G. (1989) Prog. Clin. Biol. Res. 312: 95-112; and Bazan, N. G. (2003) J. Lipid Res. 44: 2221-2233.

NPD1 has been shown to be produced by cytokine-stressed neural cells, and that the amount of DHA and NPD1 was reduced in Alzheimer disease (AD). In addition, NPD1 was previously shown to promote brain cell survival via the induction of antiapoptotic and neuroprotective gene-expression programs. See, W. J. Lukiw et al., “A role for docosahexaenoic acid-derived neuroprotection D1 in neural cell survival and Alzheimer disease,” J. Clinical Investigations, vol. 115, pp. 2774-2783 (2005). In addition, NPD1 was shown to counteract leukocyte infiltration and proinflammatory gene expression in brain ischemia-reperfusion. These findings indicate that increasing NPD1 in the brain could provide therapeutic intervention for stroke, age-related macular degeneration, spinal cord injury, and other neuroinflammatory or neurodegenerative diseases. See, N. G. Bazan et al., “Brain response to injury and neurodegeneration: endogenous neuroprotective signaling,” Ann. N. Y. Acad. Sci., vol. 1053, pp. 137-147 (2005); N. G. Bazan, “Neuroprotection D1 (NPD1): a DHA-derived mediator that protects brain and retina against cell injury-induced oxidative stress,” Brain Pathol., vol. 15, pp. 159-166 (2005); and N. G. Bazan, “Lipid signaling in neural plasticity, brain repair, and neuroprotection,” Mol. Neurobiol., vol. 32, pp. 89-103 (2005).

Pigment Epithelium-Derived Factor (PEDF)

Pigment epithelium-derived factor or pigment epithelium derived neurotropic factor (PEDF), a member of the serine protease inhibitor (serpin) family, was initially identified in the conditioned medium of human retinal pigment epithelial cells. See, J. Tombran-Tink and C. J. Barnstable (2003) Nat Rev Neurosci. 4:628-636. PEDF has been characterized, including the sequence of the purified protein and cDNA encoding the protein, and suggested to have use in the treatment of retinal diseases and tumors. See, U.S. Patent Application Publication No. 2003/0096750, and U.S. Pat. No. 6,451,763. PEDF has been shown to inhibit angiogenesis and to prevent ischemia-induced retinopathy. See, Stellmach, V., Crawford, S. E., Zhou, W., and Bouck, N. (2001), PNAS, 98: 2593-2597. PEDF is an important growth factor for retinal pigment epithelial (RPE) cells. See, U.S. Pat. No. 6,361,771 and U.S. Patent Application Publication No. 2006/0002900. It has also been shown to possess neuroprotective effect on retinal ganglion cells. See, Pang, I-H, Zeng, H., Fleenor, D. L., and Clark, A. F., (2007) BMC Neuroscience, 8: 11 (publication date 29 Jan. 2007).

PEDF has been shown to be protective against various forms of brain and retina injury, in cell cultures as well as in vivo. See, Tombran-Tink, J. (2005) Front Biosci. 10:2131-2149; Takita, H. et al. (2003) Invest. Opthalmol. Vis. Sci. 44(10):4497-4504; Ogata, N. et al. (2001)Curr. Eye Res. 22(4): 245-252; Cao, W., et al. (2001) Invest. Opthalmol. Vis. Sci. 42(7): 1646-1652. PEDF has also been found to protect against glutamate toxicity in developing primary hippocampal neurons and cultured cerebellar granule cells (See, DeCoster, M. A. et al. (1999) J. Neurosci. Res. 56(6):604-610; Taniwaki, T. et al. (1997) J. Neurochem. 68(1): 26-32; Taniwaki, T., et al. (1995) J. Neurochem. 64(6):2509-2517); protect retinal ganglion cells (See, Pang, I. H., et al. (2007) BMC Neurosci. 8:11); induce pro-survival genes through cyclic AMP-responsive element binding protein and nuclear factor kappa B (NFκB) activation in rat cultured cerebellar granule cells (See, Yabe, T., et al. (2005) Neuroscience 133(3):691-700; Yabe, T., et al. (2001) J. Biol. Chem. 276(46):43313-43319); and promote the survival and differentiation of developing spinal motor neurons (See, Houenou, L. J., et al. (1999) J. Comp. Neurol. 412(3):506-514). In addition, PEDF promotes repair of retinal cells (See, Tanimoto, S., et al. (2006) J. Med. Sci. 55(41:109-116).

### DISCLOSURE OF INVENTION

I have discovered that the unique combination of PEDF and DHA acts synergistically to enhance cell survival and decrease apoptosis. Blinding retinal degeneration (e.g., retinitis pigmentosa) is due to the progressive degeneration of photoreceptors and retinal pigment epithelial cells (RPE cells). I have discovered that PEDF (pigment epithelium-derived factor) plus DHA (docosahexaenoic acid) act synergistically in protecting the survival of the RPE cells. PEDF and DHA (a member of the omega-3 essential fatty acid family), protected human retinal pigment epithelial cells (RPE) confronted with oxidative stress. This synergistic cytoprotection is reflected in the ability of this combination to block apoptotic cell death. In addition, the combination of PEDF and DHA acted synergistically in modifying the molecular mechanisms that lead to cell death. In particular, the combined use of PEDF and DHA increased the synthesis of the important mediator neuroprotectin D1.

NPD1 was shown to be involved in the interaction between these two cell types in the retina, RPE cells and photoreceptors, when under oxidative stress. Therefore, PEDF plus DHA may be useful as a therapeutic approach in several eye diseases. In the dry form of age-related macular degeneration, apoptosis of macular photoreceptors occurs. Therefore, administering a composition comprising PEDF and DHA will elicit cytoprotection and thus halt or slow down the initiation and progression of death of the macular photoreceptors and thus decrease the amount of blindness of the patient. In the wet-form of age-related macular degeneration (AMD), one of the major problems in this form of AMD is the pathological growth of blood vessels in the retina. The combination of PEDF and DHA acts synergistically to increase the amount of NPD1 produced in the retina. This administration of this combination to the retina would increase the NPD1 concentration and thus cause a decrease in the amount of pathological angiogenesis.

The diseases retinitis pigmentosa and retinal degeneration that are caused by gene mutations can also be helped using this new combination. Although the ultimate cure will be to correct or replace the defective gene, the administration of PEDF plus DHA will slow down the initiation and progression of photoreceptor death by inhibiting the apoptotic cell death that occurs in most of those conditions. Even in glaucoma, cell death of retinal ganglion cells occurs and is a cause of some of the clinical symptoms. Again, the administration of the combination of PEDF and DHA can be used to slow down or decrease the amount of retinal cells dying. Thus, in general, symptoms of any retinal disease that involves cell death, or cell injury leading to death caused by other forms of damage, can be alleviated by administering the combination of PEDF and DHA.

The administration of the combination of PEDF plus DHA may also be protective in neurological conditions where cell damage and death takes place, such as stroke, epilepsy, Alzheimer' disease, Huntington's, Parkinson's, amyotrophic lateral sclerosis, multiple sclerosis, head injury, and spinal cord injury. Several diseases commonly described in psychiatry—depression, post-traumatic stress disorder, and schizophrenia—also involve neuronal cell death and may be alleviated by PEDF plus DHA. It is believed that the combination of PEDF and DHA administered to neural cells will increase the production of NPD1, and will thus work synergistically to promote brain cell survival, and be an effective treatment for stroke, neurotrauma, spinal cord injury, and neurodegenerative diseases, such as Alzheimer's disease, as well as in certain psychiatric illnesses.

Refractive surgery, dry eye, herpetic keratitis and several other diseases result in damage to the cornea nerves. I have also shown that the topical application of the combination of PEDF and DHA promotes cornea nerve regeneration, and thus this combination could be used to prevent the complications of refractive surgery and certain diseases, e.g., neurotrophic keratitis due to Herpes virus.

### DESCRIPTION OF DRAWINGS

FIG. 1A illustrates the experimental protocol and schedule of either A2E-induced oxidative stress or exposure to hydrogen peroxide and TNFα in serum-starved ARPE-19 cells and the timing of subsequent addition of NPD1.

FIG. 1B illustrates the appearance of Hoechst 33258 positive cells upon exposure to A2E and subsequent addition of NPD1 according to the protocol of FIG. 1A.

FIG. 1C illustrates the appearance of Hoechst 33258 positive cells upon exposure to hydrogen peroxide and TNFα and subsequent addition of NPD1 according to the protocol of FIG. 1A.

FIG. 1D depicts the potential pathway for the formation of oxiranes (epoxides) upon in the presence of oxygen and 430 nM light for 15 min.

FIG. 1E illustrates the percent distribution of oxidized A2E and its products as characterized by LC-MS-MS.

FIG. 2A illustrates the effect of A2E and NPD1 on the amount of caspase cleavage in ARPE-19 cells.

FIG. 2B illustrates the effect of A2E and NPD1 on the BAY concentration in ARPE-19 cells.

FIG. 2C illustrates the effect of A2E and NPD1 on the Bcl-2 concentration in ARPE-19 cells.

FIG. 3 illustrates the effect of various neurotrophins on NPD1 synthesis by the apical and basolateral surfaces of cultured primary human RPE cells.

FIG. 4A illustrates the cellular polarity of human retinal pigment epithelial cells and NPD1 synthesis and release upon the addition of various concentrations of CNTF and PEDF to the apical and basolaterial media.

FIG. 4B illustrates the cellular polarity of human retinal pigment epithelial cells and NPD1 synthesis and release upon the addition of various concentrations of DHA in the presence of 20 ng/ml PEDF to the apical and basolaterial media.

FIG. 4C illustrates the cellular polarity of human retinal pigment epithelial cells and NPD1 synthesis and release upon the addition of various concentrations of arachidonic acid (nM) to the apical and basolaterial media.

FIG. 4D illustrates the cellular polarity of human retinal pigment epithelial cells and NPD1 synthesis and release (as percentile distribution in cells) upon the addition of various concentrations of DHA to the apical and basolaterial media.

FIG. 4E illustrates the cellular polarity of human retinal pigment epithelial cells and NPD1 synthesis and release upon the addition of various concentrations of DHA in the presence and absence of 20 ng/ml PEDF to the apical and basolaterial media.

FIG. 5A illustrates the synergistic interaction of the addition of PEDF and DHA on NPD-1 synthesis and release in cultured ARPE-19 cells.

FIG. 5B illustrates the synergistic interaction of the addition of PEDF and DHA on the amount of apoptosis in cultured ARPE-19 cells.

FIG. 6A illustrates the effect of addition of various concentrations of DHA in the presence of 20 ng/ml PEDF on the amount of the anti-apoptotic proteins Bcl-2 and Blf-1 in ARPE-19 cells confronted with oxidative stress.

FIG. 6B illustrates the effect of addition of various concentrations of DHA in the presence of 20 ng/ml PEDF on the amount of the Bcl-family proteins BID, BAY, and BAD in ARPE-19 cells confronted with oxidative stress.

FIG. 6C illustrates the effect of addition of various concentrations of DHA in the presence of 20 ng/ml PEDF on the amount of caspase-3 expression in ARPE-19 cells confronted with oxidative stress.

### MODES FOR CARRYING OUT INVENTION

### A. PEDF and DHA Promote Nerve Regeneration

### Example 1

### Promotion of Cornea Nerve Regeneration

The effect of treatment with PEDF and DHA after either LASIK or PRK surgery was evaluated for its potential to increase the corneal nerve surface area, and thus decrease the complications seen in patients of corneal hypoesthesia and dry eye. Using a recognized experimental model, a lamellar dissection was performed on six rabbits with the use of a crescent blade and scissors creating a pocket in the cornea using techniques similar to S. Esquenazi et al., “Topical combination of NGF and DHA increases rabbit corneal nerve regeneration after photorefractive keratectomy,” Investigative Opthalmology & Visual Science, vol. 46, pp. 3121-3127 (2005). The six rabbits were divided into two groups: (1) one group treated with PEDF and DHA (100 μg), and (2) one group treated with a vehicle (saline plus albumin). In both groups, the treatment solution was delivered in collagen shields applied to the eye that would dissolve in 72 hr, and were changed twice a week. The rabbits were treated for six weeks, and were sacrificed at 8 weeks. The corneal nerves were examined at 2 and 4 weeks after surgery using confocal laser microscopy, which allows real time histology, and the total nerve surface area was calculated. After the rabbits were sacrificed, the sub-basal corneal nerves were stained with β-tubulin. As shown in Table 1, the average nerve area in the group treated with PEDF and DHA was 31.19%±0.03, while in the group treated with vehicle the average was 8.7%±0.013. This difference was highly significant, p=0.0031. These experiments show that the administration of PEDF and DHA increased the regeneration of corneal nerves as compared with untreated controls. Thus topical administration of this combination could be used to prevent the complications of refractive surgery and certain diseases, e.g., neurotrophic keratitis due to Herpes virus.

TABLE 1
Sub-Basal Corneal Nerve Area in Rabbits
Following Refractive Surgery

| | | SUB-BASAL CORNEAL |
|---|---|---|
| | RABBIT NO. | NERVE AREA (AVG %) |
| CONTROL RABBITS | R4 | 9.77 |
| | R5 | 9.20 |
| | R6 | 7.13 |
| Total Avg (SD) | | 8.7 (0.013) |
| PEDF + DHA RABBITS | R1 | 34.70 |
| | R2 | 29.37 |
| | R3 | 31.19 |
| Total Avg (SD) | | 31.19 (0.03) |

### B. PEDF and DHA Enhance Retinal Pigment Epithelial Cell Survival

### Example 2

### Materials and Methods

Human RPE cells. Cultures of retinal pigment epithelial (RPE) cells were prepared. RPE cells were seeded onto Millicell-HA culture plate inserts (Millipore, Bradford, Mass.), placed in 24-well plates, and allowed to reach confluence. Consent for use of tissue for research was obtained in compliance with Federal and State law and institutional regulations. Cultures were maintained in Chee's essential replacement medium until the experiments were performed as described in J. Hu and D. Bok (2001) Mol Vis 7:14-19. The medium includes Eagle's minimal essential medium with calcium (MEM, Irvine Scientific, Irvine, Calif.), 1% heat-inactivated calf serum (JRH Bioscience, Lennexo, Kans.), amino acid supplements, and 1% bovine retinal extract. The Millicell-HA filter inserts allow separate manipulation of the culture media bathing the apical and basal surfaces of the RPE monolayer and measurement of the transepithelial resistance (TER), which provides a measure of cell differentiation and confluency. Cultures were used for experiments once they developed a TER of at least 400 Ω·cm², as measured by an Epithelial Volt-ohmmeter (EVOM, World Precision Instruments, New Haven, Conn.).

Exposure of ARPE-19 cells to growth factors. Cells at 75-80% confluence (72 hr growth in DMEM/F12+10% FBS) were serum-starved for 2 hr before exposure to growth factors. The serum-starved cells were treated with TNF-α (Sigma-Aldrich Corporation, St. Louis, Mo.) (10 ng/ml) and H₂O₂ (600 μM) to induce oxidative stress and challenged with increasing concentrations (10, 30, 50 nM) and PEDF (Chemicon International, Temecula, Calif.) (50 ng/ml) simultaneously with oxidative stress for 4 hr before harvesting for protein analysis. Cell extracts were made and protein concentrations were adjusted by Bio-Rad protein reagent and used for Western-blot analysis. To study neuroprotection by DHA and PEDF in the oxidative stress-induced ARPE-19 cells, 72 hr cells were serum-starved for 8 hr before the introduction of oxidative stress and challenged with DHA and PEDF for 15 hr. Cells were analyzed to detect Hoechst-positive apoptotic cells.

Analysis of Bcl-2 and caspase-3 cleavage. Bcl-2 protein and caspase-3 cleavage were analyzed by Western-blot analysis. Also, ARPE-19 cells, stably transfected with a lentivirus construct containing the Asp-flu-Val caspase 3 cleavage sequence, were used. In short, 15-20 μg equivalent of each cell extract were subjected to electrophoresis on an 8-16% gel (Invitrogen Corporation, Carlsbad, Calif.) at 125 volts for 2 hr. The proteins were transferred to nitrocellulose membranes at 30 volts for 70 minutes at 4° C. The membranes were probed with primary antibodies against Bcl-2 (Santa Cruz Biotechnology, Inc., Santa Cruz, Calif.) and cleaved caspase-3 (Asp-175) (Cell Signaling) at room temperature and treated for 20 minutes with the secondary antibody, goat anti-rabbit IgG: horseradish peroxidase, and horseradish peroxidase-conjugated anti-biotin antibody, then proteins were detected by using an ECL kit (Amersham Biosciences, Buckinghamshire, United Kingdom).

Hoechst Staining. ARPE-19 cells were incubated with 2 μM Hoechst reagent dissolved in Lock's solution (Promega Corporation, Madison. Wis.) at 37° C. for 45 minutes before imaging. Cells were washed once with PBS and photographed using a Nikon DIAPHOT 200 microscope with fluorescence optics. Images were recorded by a Hamamatsu Color Chilled 3CCD camera and PHOTOSHOP 5.0 software (Adobe Systems, Mountain View, Calif.).

Exposure of RPE cells to growth factors. The upper chamber compartment was filled with 500 μl medium (bathing the apical cell monolayer surface) containing 0.1% HSA (human serum albumin), and 50 nM DHA (Sigma-Aldrich Corporation, St. Louis, Mo.), or 50 nM DHA plus added neurotrophins (10 to 200 ng PEDF or CNTF, or 20 ng of BDNF, Cardiotrophin, CNTF, FGF, GDNF, LIF, NT3, or Persephin (Alomone Labs Lt., Jerusalem, Israel)). The lower chamber was filled with 500 μl media (bathing the basal cell monolayer surface) containing 0.1% HSA. Cells were incubated for 72 hours, then apical and basal media were removed and collected for analysis. After allowing the cells to rest for at least 72 hours on fresh media, the experiments were repeated.

Lipidomic analysis. Human RPE primary cell cultures or ARPE-19 cells were separated from culture media and washed with 1 ml PBS. After addition of 1 ml methanol, cells were scraped from plates or millicell membranes and collected for lipid extraction. Media was spun-down to separate cell debris, then 350 μl were collected in 1 ml of cold chloroform:methanol (1:1). Protein precipitates were then separated by centrifugation at 1500×g (5 min, 4° C.). Lipid extracts were collected, and kept under nitrogen at −80° C. until solid-phase purification; extracts were pre-equilibrated at pH 3.0 in 10% methanol/water, then loaded to 500 mg C18 columns (Varian, Inc., Palo Alto, Calif.), and eluted with 1% methanol/ethyl acetate. Eluates were concentrated on a N2 stream evaporator. Samples were loaded to a liquid chromatograph-tandem mass spectrometer (LC-TSQ Quantum, Thermo-Finnigan; Thermo Fisher Scientific, Waltham, Mass.) installed with a Biobasic-AX column (Thermo-Hypersil-Keystone, Thermo Fisher Scientific, Waltham, Mass.) (100 mm\2.1 mm, 5-μm particle sizes), and eluted in a linear gradient [100% solution A (40:60:0.01 methanol/water/acetic acid pH 4.5) to 100% solution B (99.99:0.01 methanol/acetic acid)], at a flow rate of 300 μl/min for 30 minutes. LC effluents were diverted to an electro-spray-ionization probe (ESI) on a TSQ Quantum (Thermo-Finnigan, Thermo Fisher Scientific, Waltham, Mass.) triple quadrupole mass spectrometer. NPD1 and resolving D1 were obtained by biogenic synthesis as described in N. G. Bazan, D. L. Birkle, and T. S. Reddy (1984) Biochem Biophys Res Commun. 125:741-74, and V. L. Marcheselli et al. (2003) J. Biol. Chem. 278:43807-43817. Erratum in: (2003) J Biol Chem 278:51974. Other lipid standards (Cayman Chem. Ann Arbor, Mich.) were used for tuning and optimization and to create calibration curves. The instrument was set on full-scan mode, to detect parent ions, and selected reaction mode (SRM) for quantitative analysis, to detect product ions, simultaneously. The selected parent/product ions (m/z) and collision energy (v) obtained by running on negative ion detection mode were: 359.2/153.1/20 for NPD1, 343.2/281.2/18 for resolving D1, 351.2/195.0/22 for 20HO-LTB4 (used as internal standard), 327.2/283.3/16 for DHA, 311.3/267.3/20 Arachidonic Acid-d8 (used as internal standard, IS).

Data analysis. The data are expressed as means±SEM of three or more independent experiments; “n=” designates the amount of individual samples. The Student's t Test was used to perform statistical comparisons. Asterisks indicate p<0.05 was considered significant for all comparison. Non-statistical (NS) returns were obtained when asterisks were not indicated.

### Example 3

### A2E-mediated ARPE-19 Cell Damage is Attenuated by Neuroprotectin D1

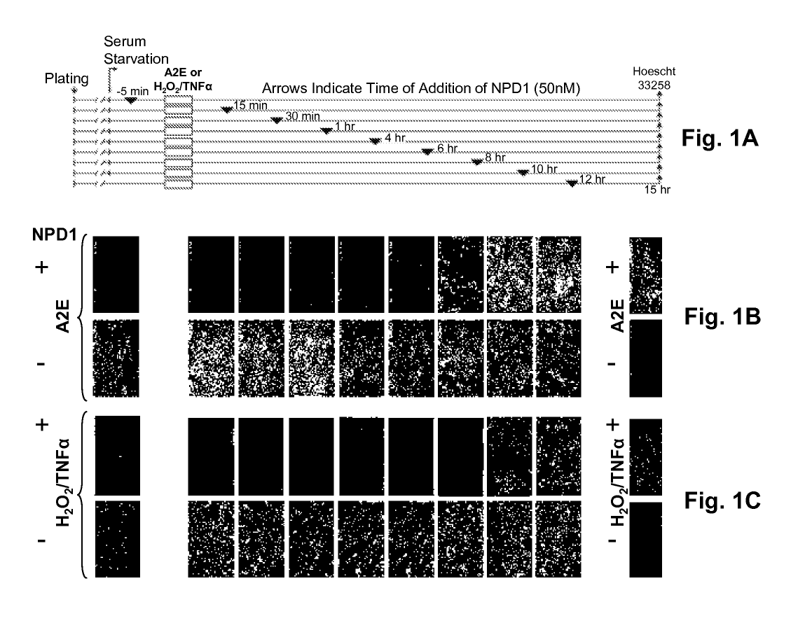

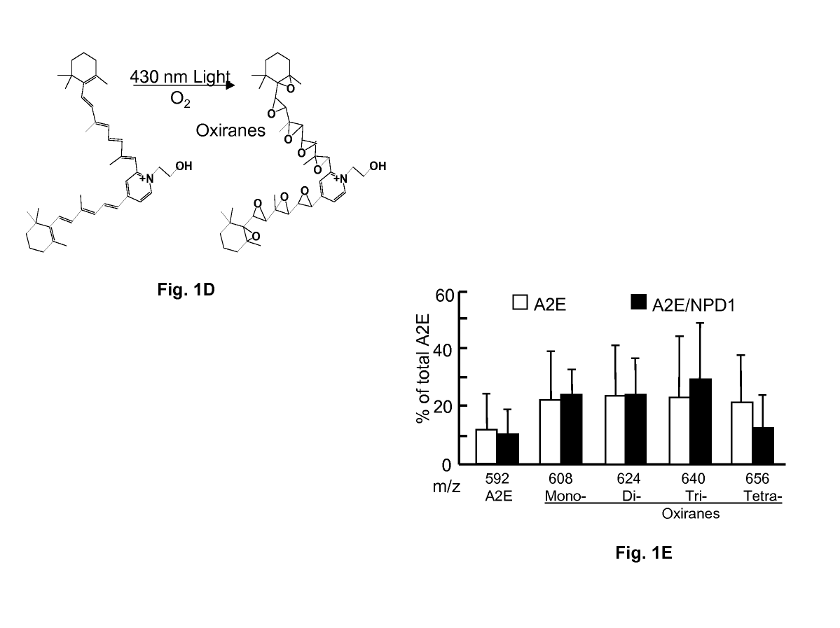

A2E, a lipofusin component, accumulates in the RPE during aging, and, in an exaggerated manner, in age-related macular degeneration (AMD) and Stargardt macular dystrophy (an early onset form of AMD). As a consequence, RPE apoptosis precedes the demise of photoreceptors. A2E-induced oxidative stress in ARPE-19 cells was shown to be inhibited by NPD1. Seventy-two (72) hours after plating, ARPE-19 cells were serum starved for 4 hours. A2E (20 μM) was added in the presence of 430 nM light and O₂ (see Materials and Methods). Other cells were exposed instead to H₂O₂/TNFα. NPD1 was added prior to, or at different times after, A2E or H₂O₂/TNFα, up to 12 hours. Time of NPD1 addition is indicated by black arrows on FIG. 1A. Hoechst 33258 was analyzed at 15 hours. FIG. 1B illustrates the appearance of Hoechst 33258 positive cells upon exposure to A2E and the effect of NPD1. FIG. 1C is similar to FIG. 1B, except that H₂O₂/TNFα was used to trigger oxidative stress. FIG. 1D depicts the formation of oxiranes (epoxides) upon exposure of ARPE-19 cells to A2E in the presence of O₂ and 430 nM light for 15 minutes followed by 60 minutes incubation in the dark. FIG. 1E shows the A2E and oxiranes characterized by LC-MS-MS (see Materials and Methods). NPD1 was a gift from Dr. Charles N. Serhan (Harvard Medical School). Data average is ±SEM (n=6) of percent distribution of oxidized A2E products.

Neuroprotectin D1 (NPD1) was cytoprotective against A2E-induced apoptosis (FIG. 1B) and unexpectedly displayed a wide time window of cytoprotection after A2E addition. Presence of NPD1 (50 nM), even 6 hours after A2E, ensures protection (FIGS. 1A and 1B). A2E-triggered oxidative stress precedes cell death. Since oxidative stress and/or inflammatory signaling are involved in early stages of retinal degenerations, oxidative stress, triggered by another experimental condition, serum starvation/H₂O₂/TNFα was explored (FIG. 1C). NPD1 also exerted protection in this experimental condition. Addition of NPD1, even eight hours after triggering oxidative stress, resulted in protection (FIGS. 1A and 1C). What was surprising was the extended time window. This was ascertained by additional experiments assessing cell viability using calcein (AM), ethidium homodimer and phase contrast microscopy that showed protection of NPD1 when added up to 15 hr after triggering oxidative stress. (Data not shown)

To study the mechanism of NPD1-mediated RPE protection against A2E oxidative stress, the possibility that A2E conversion to A2E oxiranes (epoxides) may be the target of NPD1 was tested. A2E oxiranes are the cytotoxic products that accumulate in the RPE. Multiple oxiranes (epoxides), up to nonaoxiranes, were detected by MS-MS in ARPE-19 cells exposed to light and oxygen (FIG. 1D). However, NPD1 (50 nM) did not affect this conversion (FIG. 1E).

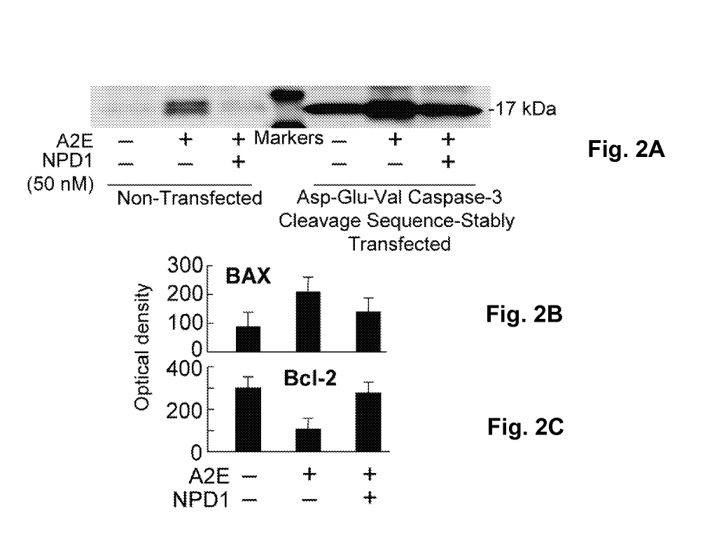

Caspase cleavage and Bcl-2 changes triggered by A2E are restored by NPD1. FIG. 2A indicates that caspase cleavage was enhanced by A2E and NPD1 attenuates this action (see Materials and Methods). FIG. 2B shows that Bax displayed a tendency to increase, whereas Bcl-2 decreases upon exposure to A2E. NPD1 (50 nM) counteracted the effect of A2E (see Materials and Methods). Data is an average of relative density detection±SEM of six individuals. Thus, NPD1 counteracted A2E-enhanced caspase-3 cleavage (FIG. 2A), showed a tendency to decrease Bax expression (FIG. 2B), and increased Bcl-2 protein expression (FIG. 2C). These findings indicate that NPD1 protects the RPE against A2E-induced apoptosis at the pre-mitochondrial level of the apoptosis cascade by altering Bcl-2 balance. This NPD1 action may be a downstream consequence of the NPD1 action on Bcl-2 proteins.

### Example 4

### Neurotrophins Promote the Synthesis and Release of NPD1 from Human RPE Cells

Human RPE cells were grown to confluence and a high degree of differentiation displaying apical-basolateral polarization as described above. These RPE cells have prominent apical microvilli, zonula occludens-positive immunoreactivity, and transepithelial resistance of at least 400 Ω·cm² (see Example 2, Materials and Methods; data not shown).

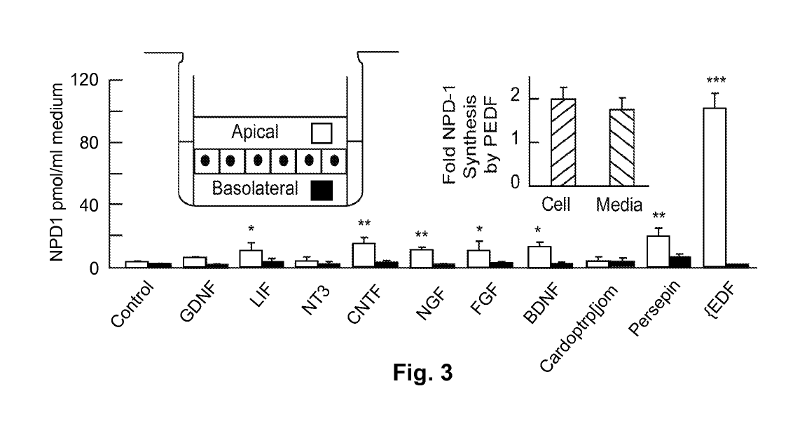

Neurotrophins activated NPD1 synthesis in cultured primary human RPE cells. FIG. 3 shows the differential ability of various growth factors to selectively release NPD1 through the apical surface of the cell. The cartoon depicts the RPE cell monolayer bath with medium on both surfaces. Growth factors (20 ng/ml) were added to the apical medium and 72 hr later, apical and basal media were collected separately and subjected to lipidomic analysis (see Materials and Methods). Each bar is an average±SEM of four or five independent wells. The insert represents net NPD1 synthesis accumulated in the cells as compared to the total media, resulting from PEDF (50 ng/ml) addition followed by lipid extraction of the cells and media 72 hr later. Increases of NPD1 in cells and media represent fold increases above those in cells incubated in the absence of growth factors. Values are averages±SEM of five independent wells. Statistical analysis shows * p<0.05, ** p<0.005, and *** p<0.0001.

FIG. 3 illustrates that several neurotrophins with bioactivities that promote neuronal and/or photoreceptor cell survival are agonists of NPD1 synthesis. Of interest is that all the neurotrophins studied, except cardiotrophin, trigger synthesis and release of NPD1 through the apical surface of the cell under the present experimental conditions. Neurotrophins were added to the upper incubation chamber (apical cellular surface). Then the upper chamber media and the lower chamber media (basolateral cellular surface) were collected separately and subjected to lipidomic analysis using LC-photodiode array-electrospray ionization-MS/MS (see Materials and Methods). Among the neurotrophins tested, pigment epithelium-derived factor (PEDF) was by far the most potent stimulator of NPD1 synthesis. PEDF, a member of the serine protease inhibitor (serpin) family, was initially identified in the conditioned medium of human retinal pigment epithelial cells cultured similarly to those used in experiments depicted in FIG. 3.

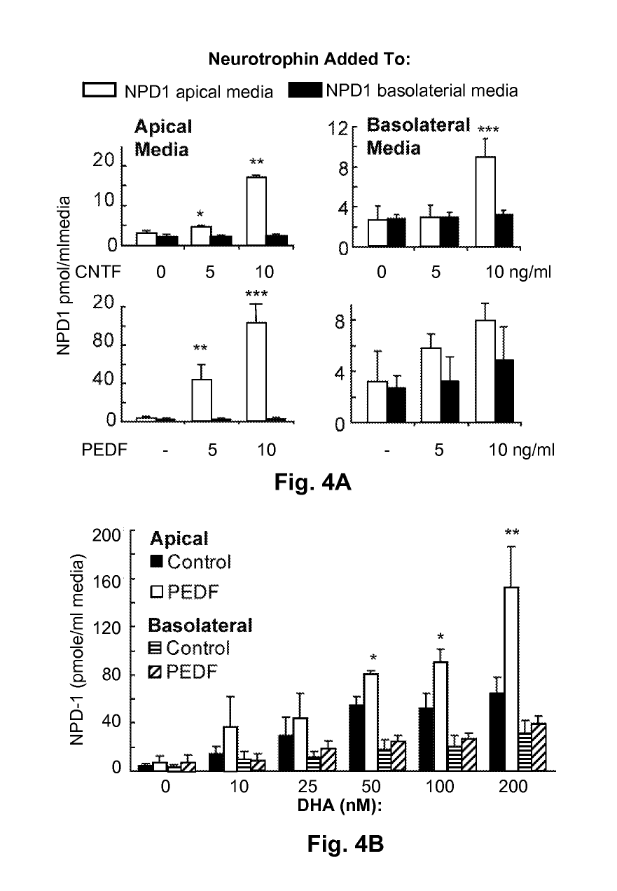

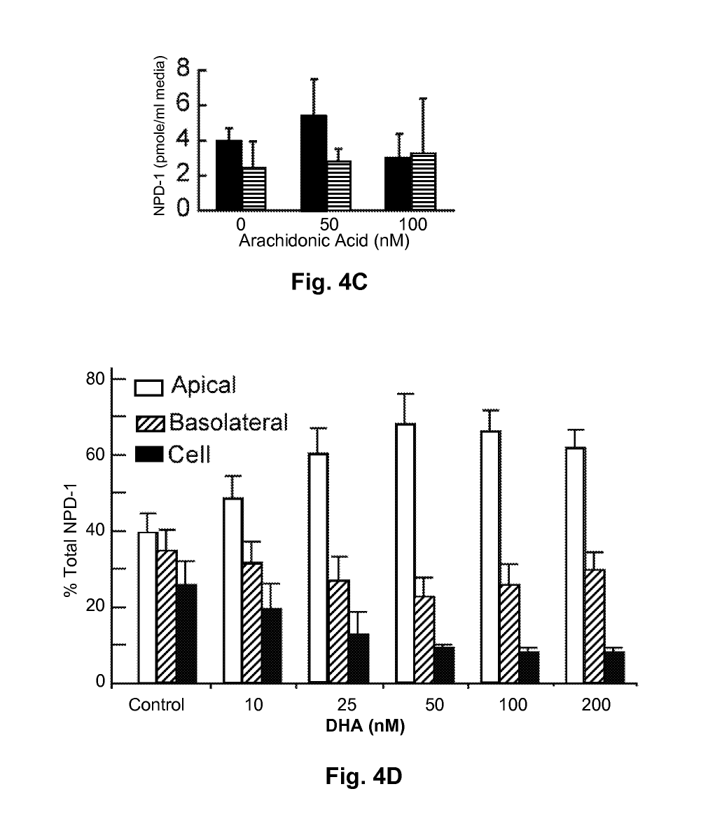

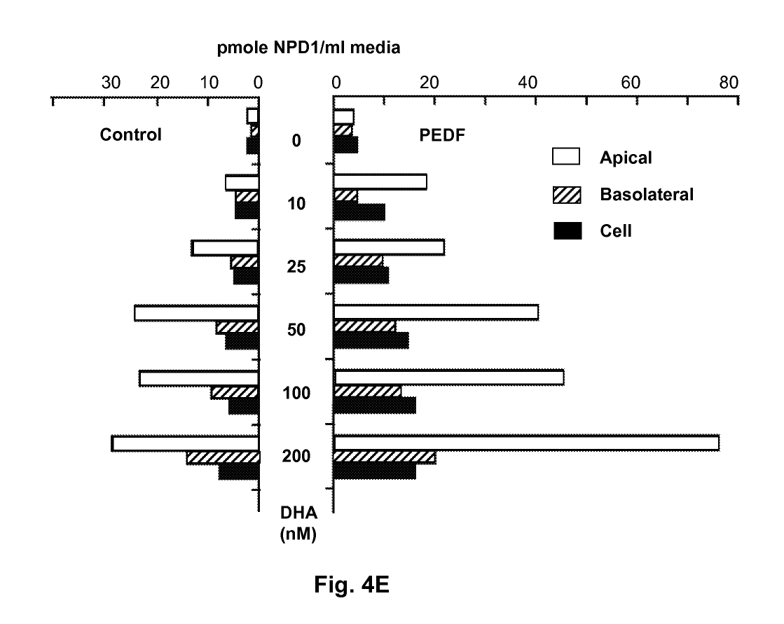

Cellular polarity of human retinal pigment epithelial cells and NPD1 synthesis and release are shown in FIGS. 4A-4E. FIG. 4A shows the concentration dependence of CNTF and PEDF activation of NPD1 release: selective response to growth factor addition to the apical RPE cell surface compared to the basal. Increasing concentrations of growth factors were added either to the apical or basal medium and 72 hr later, media were separately collected and subjected to lipidomic analysis. Relatively lower NPD1 synthesis occurred when CNTF or PEDF was added to the basal medium; however, the growth factors potently activated NPD1 synthesis and release through the apical surface when added to the apical medium. Each bar represents averages±SEM of five to seven independent wells. FIG. 4A illustrates that if PEDF or ciliary neurotrophic factor (CNTF) is added to the basal medium in increasing concentrations, they evoke much less NPD1 release on the apical side. Conversely, if these neurotrophins are added to the apical medium, they exert concentration-dependent increases in NPD1 release only on the apical side.

FIG. 4B illustrates the PEDF-induced NPD1 synthesis and release through the apical surface of RPE cells: selective potentiation by DHA. PEDF (20 ng/ml) was added to either the apical or basal medium in separate experiments and 72 hr later media were separately collected, and lipids extracted and analyzed. DHA complexed with 1% human serum albumin (Baxter, West Lake Village, Calif.) was added. Although NPD1 in the basal medium increased as the concentration of DHA was raised from 0 to 200 nM, PEDF (added to the basal medium) was unable to potentiate this action. However, DHA added to the apical medium promoted higher, concentration-dependent NPD1 synthesis and release. When the growth factor was added to the apical surface, a clear synergism in NPD1 synthesis in the presence of DHA was observed.

FIG. 4C shows that arachidonic acid, an omega-6 polyunsaturated fatty acid, when added to the apical medium under conditions similar to those of added DHA, failed to induce NPD1 synthesis. In FIG. 4D, total NPD1 percentile distribution in cells, apical and basal media is shown as a function of DHA treatment on the apical media. As shown in the figure, increasing concentration of DHA promotes significant increases of NPD1 in the apical media, reaching a maximum at 50 nM DHA. FIG. 4E shows a comparison of total distribution of NPD1 in cells, apical and basal media as a consequence of DHA concentration dependent treatment in presence or absence of 20 ng/ml PDEF. As shown in FIG. 3 and above, apical media accumulates the most NPD1, and essentially plateaus at 50 nM DHA, but treatment with PEDF, potentiates such effect. Data are average±SEM of at least three separate experiments (n=6). Statistical analysis shows * p<0.05, ** p<0.005, and *** p<0.0001.

Thus, increasing concentrations of DHA were used to further enhance PEDF-induced NPD1 synthesis and release (FIGS. 4B-E). The human RPE cells used in the present experiments have sufficient DHA in phospholipids to synthesize NPD1, as shown in controls without addition of DHA (FIGS. 4B and 4D); however, because they are in cell culture conditions, they are not undergoing photoreceptor membrane phagocytosis, and have relatively limited DHA in their phospholipids. Thus, when DHA content in the media was increased, a remarkable potentiation by PEDF of NPD1 release to the apical media was uncovered when the neurotrophin was added to the media bathing the apical cell surface (FIG. 4B). In contrast, much less NPD1 was found in the media bathing the basolateral side of the cells. Much less apical NPD1 release was observed when PEDF was applied to the media bathing the basolateral RPE surface. Regardless of the side of the cell to which PEDF is added, the amount of NPD1 release through the basolateral side was similar (FIG. 4B). Moreover, the addition of DHA to either side of the cell monolayer selectively synergized PEDF-induced NPD1 release only through the apical side. The insert (FIG. 4C) shows that added arachidonic acid even in the presence of PEDF did not stimulate NPD1 synthesis.

Next the net synthesis of NPD1 in the RPE cells was examined and compared this with the content of this lipid mediator in the culture media by bathing the apical and basal surfaces of these cells with increasing concentrations of DHA, the NPD1 precursor (FIG. 4E). The cellular NPD1 content decreased as a function of DHA concentration from 25%, when no DHA was added, down to 7% in the presence of 200 nM DHA (FIG. 4D), but concomitantly in the apical medium it increased almost proportionally. NPD1 in the apical medium accounted for 40% of its total in the absence of added DHA, and increased step-wise as DHA rose from 10 to 50 nM, without further increases at higher concentrations (FIGS. 4D and 4E). Although DHA alone does cause NPD1 synthesis, most of the newly formed NPD1 is recovered from the apical medium; much less appeared in the basal medium (FIG. 4D). The addition of PEDF (50 ng/ml) promoted an enhancement of this profile, whereby the cellular NPD1 content increased in the apical medium, as DHA was increased from 0 to 200 nM (FIG. 4B). The polarity of actions for the neurotrophin-mediated response raises the possibility that NPD1 may function, at least in part, as an autocrine and paracrine signal on cells that surround the interphotoreceptor matrix, namely the photoreceptor cells and Müller cells. Moreover, the apical side of the RPE participates in the recognition and shedding of photoreceptors during outer segment phagocytosis Furthermore, interphotoreceptor matrix proteins may be acceptors of NPD1, to facilitate its diffusion and to target it to cellular site(s) of action.

### Example 6

### DHA and PEDF Provide Cytoprotection Synergistic-Ally when RPE Cells are Confronted with Oxidative Stress

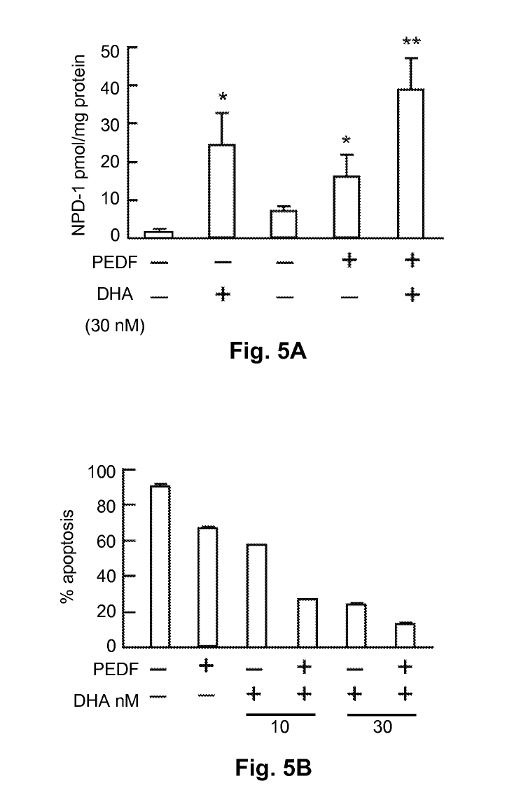

To study the downstream signaling of NPD1 synthesis induced by PEDF, ARPE-19 cells were used since, when exposed to oxidative stress, ARPE-19 cells respond with significant NPD1 synthesis and display NPD1-mediated cytoprotection. DHA was shown to potentiate PEDF bioactivity of cultured ARPE-19 cells. FIG. 5A illustrates the synergistic induction of NPD1 synthesis. Data shown are average±SEM (n=5). Asterisks indicate significance of the Student's t Test: * p<0.05; ** p<0.001. In FIG. 5B, decreased apoptosis by increasing added DHA to 30 nM in the presence of PEDF is shown. Representative Hoechst staining of experiment shown in FIG. 5B supported the results (data not shown). ARPE-19 cells, like human RPE cell primary cultures (FIG. 5B), were found to up-regulate NPD1 synthesis in the presence of PEDF (FIG. 5A). Moreover, significant cytoprotection and enhanced NPD1 formation occurred synergistically when PEDF was added along with DHA under conditions of oxidative stress-induced apoptotic cell death triggered by serum starvation/H₂O₂/TNFα (FIG. 5B).

### Example 7

### DHA and PEDF Synergistically Stimulate Anti-Apoptotic Compounds During Oxidative Stress

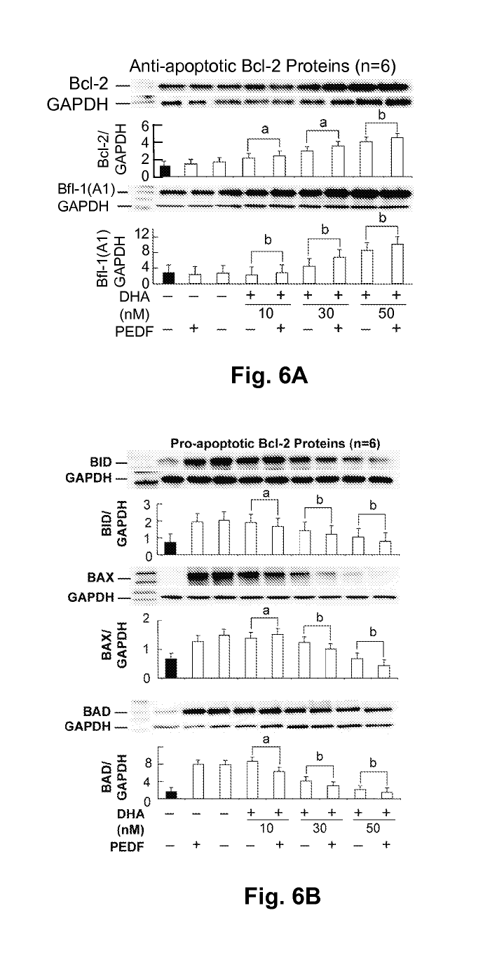

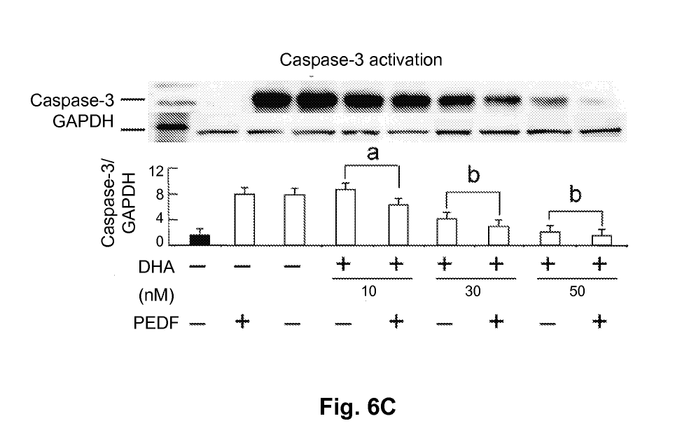

Because the initiation and amplification of the premitochondrial apoptotic cascade involves the Bcl-2 family of proteins, the expression of the anti-apoptotic proteins Bcl-2 and Bfl-1, and of the pro-apoptotic proteins Bid, Bax, and Bad, were studied during serum starvation H₂O₂/TNFα-induced ARPE-19 cell death. FIGS. 6A-6c illustrate that the Bcl-2 family proteins and caspase-3 expression are mediated by DHA and PEDF when ARPE-19 cells are confronted with oxidative stress. FIG. 6A shows the synergistic enhancement of Bcl-2 and Blf-1/A1 anti-apoptotic proteins as DHA is increased from 10 to 50 nM in the presence of PEDF. FIG. 6B shows that Bid, Bax and Bad decreased upon increasing DHA concentration in the presence of PEDF. Data represents the densitometry ratios of Bad, Bax, and Bid to GAPDH. Black bar (in FIG. 6A) represent cells not exposed to oxidative stress. Open bars (in FIGS. 6A and 6B) represent cells exposed to oxidative stress. FIG. 6C shows the converse changes in caspase-3 activation. Each bar represents average±SEM of 9 to 12 independent wells: a. not significant; b. p<0.0001.

Increasing the concentration of DHA from 10 to 50 nM was shown to up-regulate Bcl-2 and Bfl-1 protein expression (FIG. 6A). Although PEDF alone was unable to alter the expression of pro- and anti-apoptotic proteins when added with DHA during serum starvation/H₂O₂/TNFα-induced oxidative stress, it did potentiate the expression of these proteins with concomitant NPD I synthesis in the presence of DHA (FIG. 6A). Pro-apoptotic protein expression under these experimental conditions in the presence of DHA and PEDF underwent opposite changes. Bid, Bax, and Bad expressions were enhanced by oxidative stress and DHA decreased their expressions, whereas PEDF potentiated this action (FIG. 6B).

The marked increase in the numbers of Hoechst-positive ARPE-19 cells during oxidative stress correlated well with caspase-3 cleavage (FIG. 6C). Effector caspase-3 downstream of cytochrome-c release from mitochondria and apoptosome activation progressively decreased when cells were exposed to 10 to 50 nM DHA; PEDF potentiated this action (FIG. 6C). A remarkable synergy between PEDF and DHA was demonstrated with enhanced cytoprotection, up-regulation of NPD I synthesis, enhancement of anti-apoptotic protein expression, down-regulation of pro-apoptotic protein expression, and caspase-3 cleavage.

The data presented here indicate that apoptosis triggering of the bispyridinium bisretinoid A2E is markedly attenuated, displaying a wide window of cytoprotection by NPD1 in ARPE-19 cells. In contrast, NPD1 did not affect the photooxidation of A2E as measured by the conversion of A2E into A2E oxiranes. The present observations support the notion that NPD1 elicits a specific action, other than antioxidant activity, to counteract A2E-induced apoptosis in RPE cells. Moreover, the wide window of NPD1 cytoprotection against A2E is similar to that exerted by serum-deprivation/H₂O₂/TNFα, indicating that the bioactivity of this lipid mediator may act at initial checkpoints of cell apoptosis. PEDF and NPD1 are antiangiogenic factors; thus, the synergy reported here may be relevant to the management of pathoangiogenesis in macular degeneration and tumors.

In addition, PEDF was shown to be a NPD1 synthesis agonist and selective activator of the apical efflux of the lipid mediator in human RPE cells in monolayer cultures. Also, DHA greatly potentiates PEDF-induced RPE cytoprotection against oxidative stress, with concomitant NPD1 formation. The synergy with PEDF and DHA indicates that the availability of the NPD1 initial precursor is critical for its synthesis.

The regulation of apoptosis involves multiple checkpoints. The ability of DHA to potentiate PEDF bioactivity on expression of the Bcl-2 family of proteins indicates that the pre-mitochondrial stage of the apoptotic cascade checkpoint is involved in the observed cytoprotection, with concomitant NPD1 formation. The ability of neurotrophies to promote cell survival through NPD1 in the RPE cell, as described here, is also highly relevant to the response of the nervous system to injury and neurodegeneration. Neurotrophins, as agonists of NPD1 synthesis from DHA, may promote signaling integration for cell survival. In fact, NPD1 fosters homeostatic regulation of cell integrity during photoreceptor cell renewal. The regulation of pro- and anti-apoptotic proteins during the window of protection shown here will contribute to further define NPD1 survival bioactivity. These events are clinically significant because they will contribute to the exploration of therapeutic interventions for neurodegeneration, particularly retinal degenerative diseases.

As used in the specification and claims, the term “PEDF” refers to pigment epithelium-derived factor (also called pigment epithelium derived neurotrophic factor), its truncated version PEDF-BH, and functionally equivalent proteins. As used in the specification and claims, the term “DHA” refers to docosahexaenoic acid, alkali metal salts thereof, and alkyl esters thereof.

As used in the specification and claims, an “effective amount” of the PEDF plus DHA composition is an amount that is sufficient to increase the synthesis of NPD 1 or the cell survival to a significant degree. In the case of addition to the corneal, the “effective amount” of PEDF and DHA is an amount sufficient to increase the corneal nerve area to a significant degree. Significance for this purpose is determined as the P<0.5 level, or by such other measure of statistical significance as is commonly used in the art for a particular type of experimental determination. The dosage ranges for the administration of PEDF plus DHA are those that produce the desired effect. Generally, the dosage will vary with the age and condition of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the improvement in the symptoms of the disease being treated—e.g., the increase in corneal nerve area can be monitored by methods well known to those in the field, and the decrease of cell death in the retinal or neural cells can be monitored by well-known methods, shown in this application or in the references cited therein. Moreover, the PEDF plus DHA can be applied in pharmaceutically acceptable carriers known in the art. The application depends on the location of the tissue being treated. For treatment of the cornea, the preferred application is topical. For the treatment of retinal cells, the preferred application is intraocularly. For the treatment of brain neural cells, the preferred application is intracerebrally.

Controlled delivery may be achieved by admixing the combination of PEDF and DHA with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of PEDF plus DHA may be controlled by altering the concentration of the macromolecule.

Another method for controlling the duration of action comprises incorporating the PEDF and DHA into particles of a polymeric substance such as a polyester, peptide, hydrogel, polylactide/glycolide copolymer, or ethylenevinylacetate copolymers. In addition, the combination could be delivered to the eye (intraocular delivery) and brain (intracerebral delivery) by use of encapsulated PEDF and DHA in slow delivery capsules, e.g., a liposome. For example in the eye, these capsules could be implanted in the vitreous body in front of the retina.

In addition, the RPE cell is a versatile system being used as an implantable system into the brain in neurodegenerative diseases. There have been attempts to culture RPE cells on biodegradable polymers and on amniotic membranes for transplantation. See, G. G. Giordano et al., “Retinal pigment epithelium cells cultured on synthetic biodegradable polymers,” J. Biomed. Mater. Res., vol. 34, pp. 87-93 (1997); and U.S. Patent Application Publication No. 2006/0002900. Also, genetically modified RPE cells have been implanted for enhancing visual function in dystrophic rats. See, R. D. Lund et al., “Subretinal transplantation of genetically modified human cell lines attenuates loss of visual function on dystrophic rats, PNAS, vol. 98, pp. 9942-9947 (2001). Another neurotrophic factor (ciliary neurotrophic factor) has been encapsulated in RPE cells for use in neurodegenerative diseases. The RPE cells were proposed for both encapsulated and un-encapsulated cell-based delivery technology, including the genetic modification to secrete an efficacious quantity of a growth factor, including PEDF. See, U.S. Pat. No. 6,371,771. I would propose adding the DHA to these cells for the above treatments intraocularly and intracerebrally. The RPE cells could also be pre-incubated with PEDF and DHA to increase the production of NPD1, and then delivered to the eye or brain.

For administration to the cornea, the PEDF and DHA can be administered using a collagen shield or contact lens that is somewhat absorbent of the complex, e.g., Soft Shield Collagen Shield, 72-hour (Oasis Medical Inc., Glendora, Calif.), hilafilcon B soft 2-week contact lens (Bausch & Lomb, Rochester, N.Y.), and Night and Day soft contact lenses (Ciba Vision, Duluth, Ga.). The shield or lens can be made of any hydrophilic transparent polymer, such as poly-hydroxyethylmethacrylate hydrogel, ethoxy ethyl methacrylate hydrogel, methacrylic acid, n-vinylpyrrolidinone, siloxane hydrogel, polydimethylsiloxane polyols, perfluoropolyethers, dimethylacrylamide, methyl methacrylate, and fluorosiloxane hydrogel, as discussed in P. C. Nicolson et al., “Soft contact lens polymers: an evolution,” Biomaterials, vol. 22, pp. 3273-3283 (2001). For additional methods of delivery for a growth factor and DHA to a cornea, see International Publication Number WO 2006/044090.

The present invention provides a method of treating or attenuating the symptoms of a patient with a disease that causes retinal cell degeneration or neurodegeneration. The present invention also provides a method of treating or attenuating the symptoms of dry eye or other neurotrophic keratopathies resulting from some disruption to the corneal nerve supply, comprising topically administering to a patient who has an injured cornea (e.g., one who has undergone PRK or LASIK) an effective amount of PEDF plus DHA. The term “attenuate” refers to a decrease or lessening of the symptoms or signs of the underlying disease.

The complete disclosures of all references cited in this application are hereby incorporated by reference. Also incorporated by reference are the following publications which are not prior art to this application: P. K. Mukherjee et al., “Neurotrophies enhance retinal pigment epithelial cell survival through neuroprotectin D1 signaling,” PNAS, vol. 104, pp. 13152-13157 (2007); P. K. Mukherjee et al., “Photoreceptor outer segment phagocytosis attenuates oxidative stress-induced apoptosis with concomitant neuroprotectin D1 synthesis,” PNAS, vol. 104, pp. 13158-13163 (2007); and N. G. Bazan, “Homeostatic regulation of photoreceptor cell integrity: significance of the potent mediator neuroprotectin D1 biosynthesized from docosahexaenoic acid: the Proctor Lecture,” Invest Ophthahnol Vis Sci, vol. 48, pp. 4866-81 (2007). In the event of an otherwise irreconcilable conflict, however, the present specification, including this narrative and all attached pails, shall control.

I claim:

1. A sterile medical composition comprising pigment epithelium-derived factor (PEDF) and docosahexaenoic acid (DHA).

2. The medical composition of claim 1, wherein said DHA is bound to albumin.

3. An implantable cell culture device, the device comprising a semipermeable membrane permitting the diffusion of pigment epithelium-derived factor (PEDF) and docosahexaenoic acid therethrough; and retinal pigment epithelial cells that are genetically engineered to produce PEDF and that are infused with docosahexaenoic acid, both disposed within the semipermeable membrane.

4. A method for inhibiting retinal degeneration, comprising implanting into the eye of a recipient host with retinal degeneration an effective amount of the medical composition of claim 1 or the implantable cell culture device of claim 3.

5. A method for inhibiting retinal degeneration as in claim 4, wherein said retinal degeneration is associated with one or more diseases selected from the group consisting of age-related macular degeneration, retinitis pigmentosa, and glaucoma.

6. A method for inhibiting neural degeneration, comprising implanting into the neural tissue of a recipient host with neural degeneration an effective amount of the medical composition of claim 1 or the implantable cell culture device of claim 3.

7. A method as in claim 6, wherein the neural degeneration is associated with one or more diseases selected from the group consisting of stroke, epilepsy, Alzheimer's disease, Huntington's disease, Parkinson's disease, amyotrophic lateral sclerosis, multiple sclerosis, head trauma, spinal cord injury, depression, post-traumatic stress disorder, and schizophrenia.

8. A method to enhance nerve re-generation in an injured cornea, said method comprising topically administering to the injured cornea an effective of amount of the medical composition of claim 1.

9. The method as in claim 8, wherein said cornea is injured by a cause selected from the group consisting of trauma, photorefractive keratectomy (PRK), laser in situ keratomilieusis, chemical burn, congenital corneal neuropathy, acquired corneal neuropathy, dry eye, and herpetic keratitis.

10. A composition comprising a mixture of an effective amount of epithelium-derived factor, an effective amount of docosahexaenoic acid, and a pharmaceutically acceptable carrier; wherein said composition is sterile.

11. A composition as in claim 10, wherein said docosahexaenoic acid is bound to albumin.

12. An article of manufacture comprising a sterile covering adapted to protect an injured human cornea in vivo; wherein said covering comprises an effective amount of a composition as recited in claim 10; and wherein said article is adapted to release said composition over time when in contact with a cornea in vivo.

13. An article of manufacture as recited in claim 12, wherein said covering comprises collagen.

14. An article of manufacture as recited in claim 12, wherein said covering comprises a transparent polymer selected from the group consisting of poly-hydroxyethylmethacrylate hydrogel, ethoxy ethyl methacrylate hydrogel, methacrylic acid, n-vinylpyrrolidinone, siloxane hydrogel, polydimethylsiloxane polyols, perfluoropolyethers, dimethylacrylamide, methyl methacrylate, and fluorosiloxane hydrogel.

15. An article of manufacture as recited in claim 12, wherein said covering additionally comprises a macromolecule selected from the group consisting of polyesters, polyamino acids, polyvinyl pyrrolidone, ethylenevinylacetate, methylcellulose, carboxymethylcellulose, prolamine sulfate, and lactide/glycolide copolymers; wherein said macromolecule will alter the rate of release of said composition when said article is in contact with a cornea in vivo, as compared with the rate of release from an otherwise identical article of manufacture lacking said macromolecule.
